(19) United States
(12) Patent Application Publication
(10) Pub. No.: US 2008/0177161 A1
(43) Pub. Date: Jul. 24, 2008

(54) Reflective infrared ray artery waveform collector

(76) Inventor: Chunhui Nan, (US)

Correspondence Address: PRO-TECHTOR INTERNATIONAL SERVICES, 20775 NORADA CT., SARATOGA, CA, 95070, US

(21) Appl. No.: 11/986,604

(22) Filed: Nov. 23, 2007

(30) Foreign Application Priority Data
Jan. 19, 2007 (TW) 096201113

(51) Int. Cl. A61B 5/1455

(52) U.S. Cl. 600/310

(57) ABSTRACT

A reflective infrared ray artery waveform collector comprises a housing module, a collecting module for collecting the artery waveforms, and a transmission module. The collecting module is disposed in the housing module and includes a circuit board, and a microprocessor and a reflective infrared ray sensor, both of which are electrically connected to the circuit board respectively, and the reflective infrared ray sensor includes an emission element, at one side of which is mounted a receiving element, thereby by using the emission element, an infrared ray reflects to a vessel of human body, and then by using the receiving element, an infrared ray signal reflected from the vessel is received for accessing the blood information of human body, calculated by the microprocessor and then transmitted to a blood analyzer by the transmission module for being transformed into an artery waveform.

### BACKGROUND OF THE INVENTION

1. Field of the Invention

The present invention relates to an artery waveform collector, and more particularly to a reflective infrared ray artery waveform collector that may utilize an infrared ray sensor to collect the artery waveform.

2. Description of the Prior Arts

In current days, people are concerned with health issue more and more, especially cardiovascular disease has become the second killer to human life. Nevertheless, most of people neglect the importance of health examination due to the hustle and bustle life. To enhance the desire of health examination, providing a specific examining instrument with cost-saving and efficiency-enhancing features, such as pulse sensor, has become an inevitable tendency in healthcare field.

The present invention has arisen to mitigate and/or obviate the afore-described disadvantages.

### SUMMARY OF THE INVENTION

The primary object of the present invention is to provide a reflective infrared ray artery waveform collector that may utilize an infrared ray sensor to collect the artery waveform.

The secondary object of the present invention is to provide a reflective infrared ray artery waveform collector that may lower the collecting cost economically and collect the desired pulse waveform easily.

Another object of the present invention is to provide a reflective infrared ray artery waveform collector that may easily capture any desired pulse/bleeding waveform, such as artery, radial artery, cerebral artery, temple and the like, utilizing widely.

In accordance with one aspect of the present invention, there is provided a reflective infrared ray artery waveform collector comprising a housing module, a collecting module for collecting the artery waveform, as well as a transmission module, wherein the collecting module is disposed in the housing module and includes a circuit board, and a microprocessor and a reflective infrared ray sensor, both of which are electrically connected to the circuit board respectively, wherein the reflective infrared ray sensor includes an emission element, at one side of which is mounted a receiving element; a transmission module affixed in the housing module and electrically connected with the collecting module; thereby by using the emission element, an infrared ray reflects to a vessel of human body, and then by using the receiving element, an infrared ray signal reflected from the vessel is received for accessing the blood information of human body, calculated by the microprocessor and then transmitted to a blood analyzer by the transmission module for being transformed into an artery waveform.

The present invention will become more obvious from the following description when taken in connection with the accompanying drawings, which show, for purpose of illustrations only, the preferred embodiment in accordance with the present invention.

### BRIEF DESCRIPTION OF THE DRAWINGS

FIG. 1 is a block diagram illustrating the function of a reflective infrared ray artery waveform collector according to a first embodiment of the present invention;

FIG. 2 is a perspective diagram illustrating the exploded components of the reflective infrared ray artery waveform collector according to the first embodiment of the present invention;

FIG. 3 is a cross sectional diagram illustrating the assembly of an infrared ray sensor according to the first embodiment of the present invention;

FIG. 4 is a perspective diagram illustrating the operation of the reflective infrared ray artery waveform collector according to the first embodiment of the present invention;

FIG. 5 is a block diagram illustrating the function of a reflective infrared ray artery waveform collector according to a second embodiment of the present invention;

FIG. 6 is a perspective diagram illustrating the exploded components of the reflective infrared ray artery waveform collector according to the second embodiment of the present invention;

FIG. 7 is a perspective diagram illustrating the operation of the reflective infrared ray artery waveform collector according to the second embodiment of the present invention;

FIG. 8 is a partial perspective diagram illustrating the exploded components of the reflective infrared ray artery waveform collector according to the second embodiment of the present invention.

### DETAILED DESCRIPTION OF THE PREFERRED EMBODIMENT

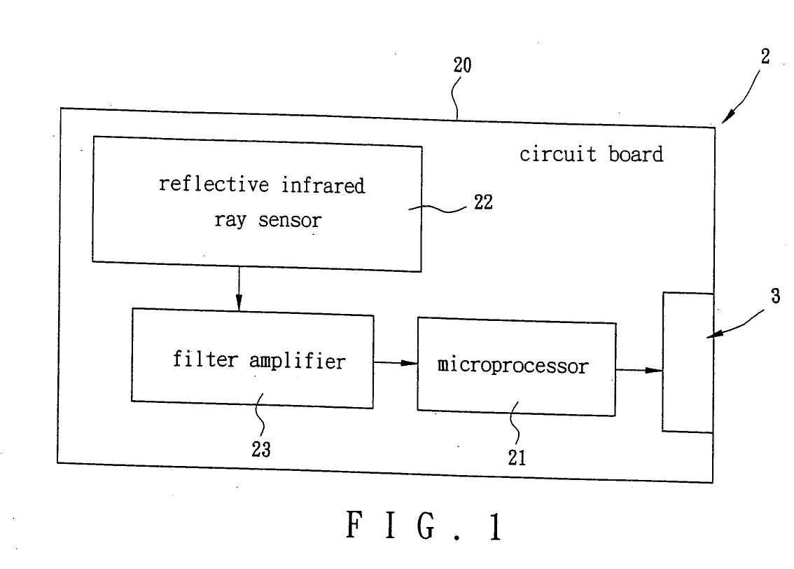

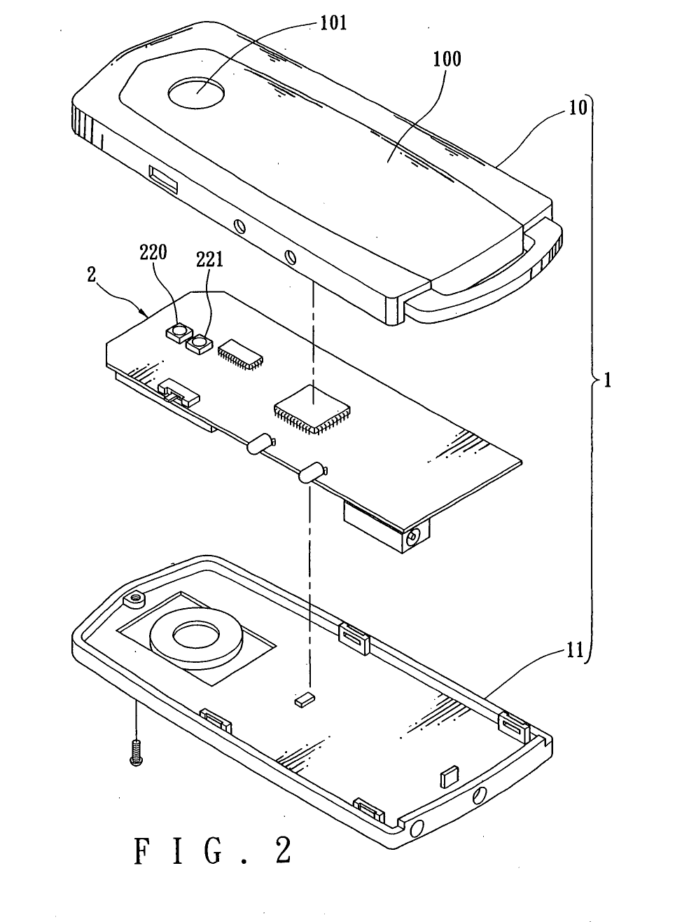

Referring to FIGS. 1-4, a reflective infrared ray artery waveform collector in accordance with a first embodiment of the present invention comprises a housing module 1, a collecting module 2 for collecting the artery waveforms, as well as a transmission module 3, wherein

the housing module 1 is comprised of an upper housing 10 and a matching lower housing 11, both of which may be assembled together in different manners, and the upper hosing 10 includes a waiting area 100, at the front end of which is provided with a transparent zone 101.

Besides, the collecting module 2 is disposed in the housing module 1 and includes a circuit board 20, a microprocessor 21, a reflective infrared ray sensor 22 and a filter amplifier 23, wherein the microprocessor 21, the infrared ray sensor 22 and the filter amplifier 23 are electrically connected to the circuit board 20 respectively, and the infrared ray sensor 22 may be visible through the transparent zone 101.

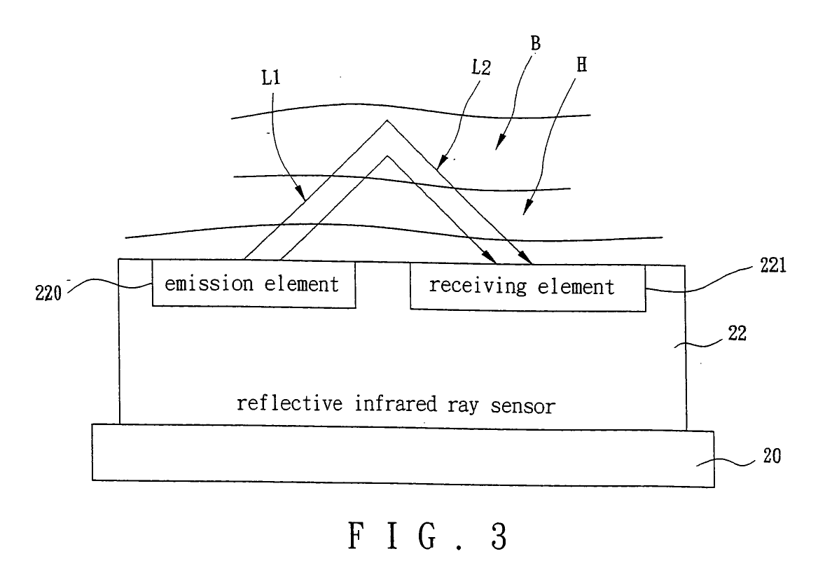

As illustrated in FIG. 3, the infrared ray sensor 22 includes an emission element 220, at one said of which is mounted a receiving element 221, wherein the emission element 220 may be an infrared ray LED and includes infrared ray with around 900-960 nanometer of wave length, and the receiving element 221 may be an infrared ray LED or triode. While the emission element 220 emits an infrared ray L1 to a vessel B of an human body, the infrared ray L1 is reflected to become an infrared ray signal L2 having the related blood information therein and then received by the receiving element 221 so as to capture blood information of human body H, and to filter and amplify it by way of the filter amplifier 23.

The transmission module 3 is affixed in the housing module 1 and electrically connected with the collecting module 2, wherein it may be a wireless or wired transmission module. For the wireless transmission module, it is a bluetooth module, yet for the wired transmission module, it may be a USB transmission line. As shown in FIG. 3, the transmission line 3 is a wireless transmission module.

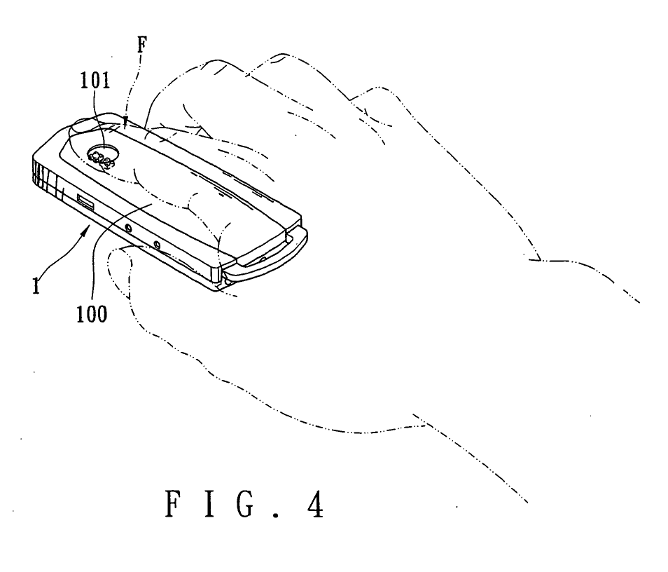

Referring further to FIGS. 3 and 4, as the user places his finger F in the waiting zone 100 of the housing module 1, the emission element 220 in the lower end of the transparent zone 101 reflects the infrared ray L1 to the vessel B of human body H, and then by using the receiving element 221, the infrared ray signal L2 reflected from the vessel B is received for accessing the blood information of human body H. Thereafter, the signal is amplified by the filter amplifier 23, calculated by the microprocessor 21, and then transmitted to a blood analyzer (not shown) by the transmission module 3 for being transformed into an artery waveform, wherein the blood analyzer may be cell phones, computers or PDAs with or without bluetooth function which are installed an analyzing software related to artery waveform figure of human body or bleeding parameters therein.

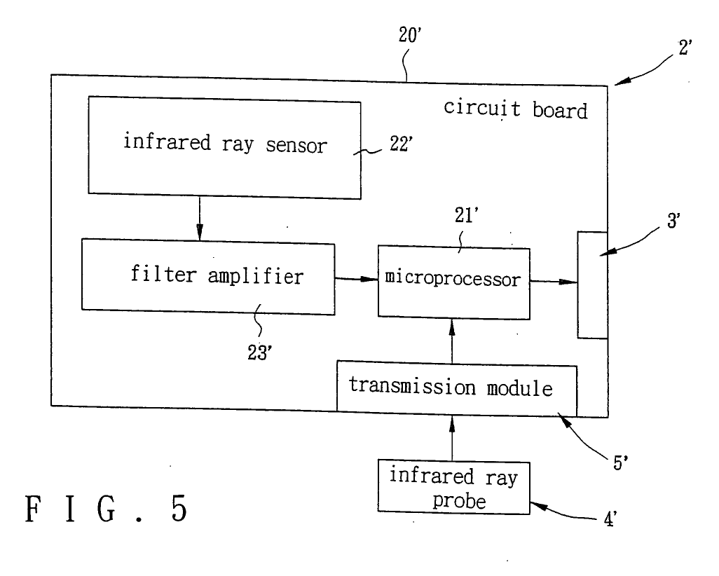

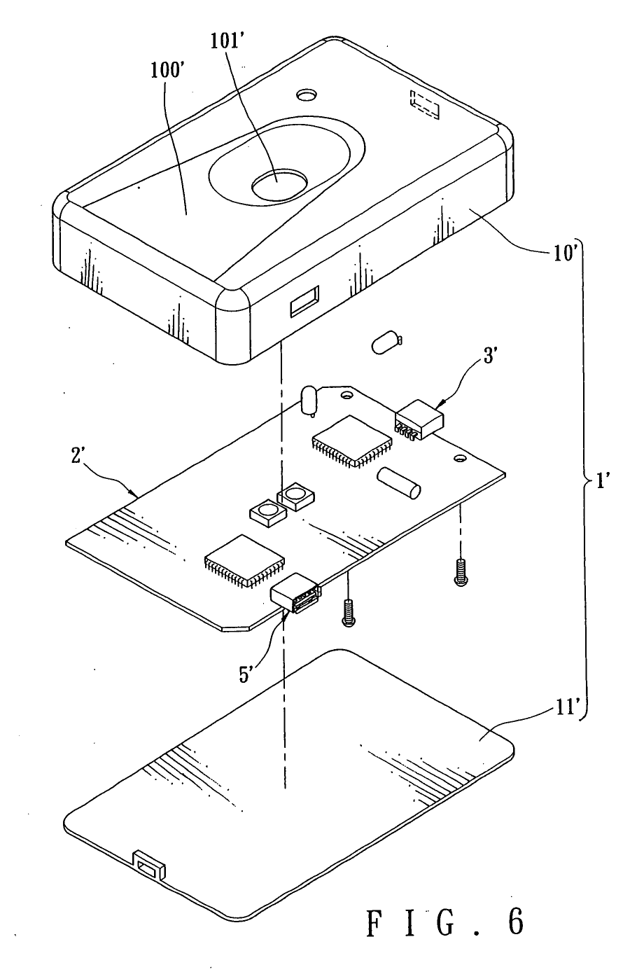

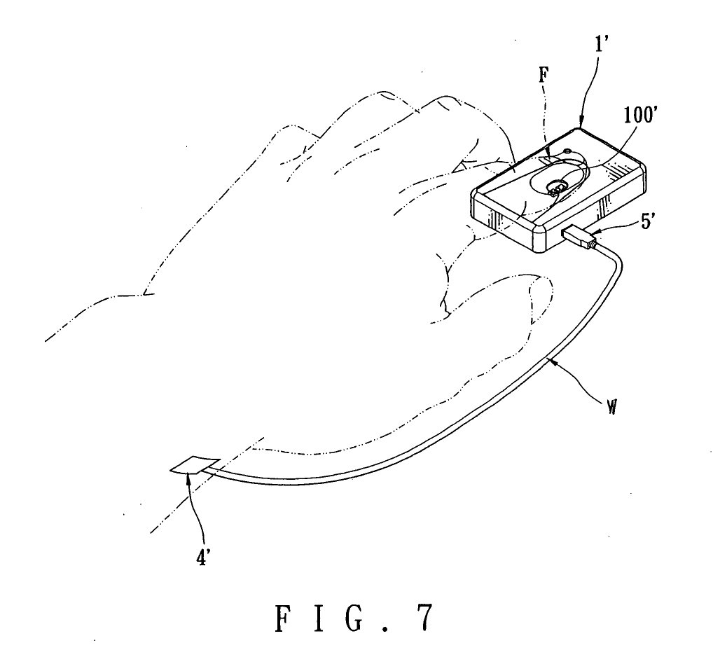

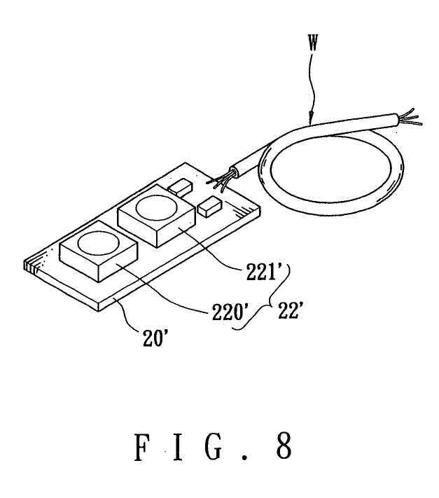

As shown in FIGS. 5-8, a reflective infrared ray artery waveform collector in accordance with a second embodiment of the present invention comprises a housing module 1′, a collecting module 2′ for collecting the artery waveforms, as well as a transmission module 3′, wherein the housing module 1′ is comprised of an upper housing 10′ and a matching lower housing 11′, and the collecting module 2′ includes a circuit board 20′, a microprocessor 21′, an infrared ray sensor 22′ (having an emission element 220′ and a receiving element 221′ therein) and a filter amplifier 23′.

On the other hand, the reflective infrared ray artery waveform collector according to the second embodiment of the present invention also comprises an infrared ray probe 4′ and a transmission module 5′, wherein one end of the transmission module 5′ is electrically connected with the collecting module 2′, and the infrared ray probe 4′ is electrically connected to another end of the transmission module 5′ by means of a transmission line W. Therefore, as placing the finger F to a waiting zone 100′ of the housing module 1′ for capturing the artery, and as the infrared ray probe 4′ is attached to the radial artery of human body, the bleeding speed may be measured by using the distance between the finger artery and the radial artery.

It can be clearly seen from the preceding accounts on the features of the present invention that the reflective infrared ray artery waveform collector of the present invention has the following advantages:

1. The present invention is structured simply, lowering the collecting cost economically and collecting the desired pulse waveform easily.

2. The present invention may easily capture any desired bleeding waveform, such as artery, radial artery, cerebral artery, temple and the like, expanding usage.

The invention is not limited to the above embodiment but various modifications thereof may be made. It will be understood by those skilled in the art that various changes in form and detail may made without departing from the scope and spirit of the present invention.

What is claimed is:

1. A reflective infrared ray artery waveform collector comprising:
a housing module;
a collecting module for collecting the artery waveforms disposed in said housing module and including a circuit board, and a microprocessor and a reflective infrared ray sensor, both of which are electrically connected to said circuit board respectively, wherein said reflective infrared ray sensor includes an emission element, at one side of which is mounted a receiving element;
a transmission module affixed in said housing module and electrically connected with said collecting module;
thereby by using said emission element, an infrared ray reflects to a vessel of human body, and then by using said receiving element, an infrared ray signal reflected from said vessel is received for accessing the blood information of human body, calculated by said microprocessor and then transmitted to a blood analyzer by said transmission module for being transformed into an artery waveform.

2. The reflective infrared ray artery wave as claimed in claim 1, wherein said housing module is comprised of an upper housing and a matching lower housing.

3. The reflective infrared ray artery wave as claimed in claim 1, wherein said emission element may be an infrared ray LED.

4. The reflective infrared ray artery wave as claimed in claim 1, wherein said emission element includes infrared ray with around 900-960 nanometer of wave length.

5. The reflective infrared ray artery wave as claimed in claim 1, wherein said receiving element may be an infrared ray LED or triode.

6. The reflective infrared ray artery wave as claimed in claim 1, wherein said collecting module includes a filter amplifier for amplifying the signal of the blood information electrically connected to said circuit board.

7. The reflective infrared ray artery wave as claimed in claim 1, wherein said transmission module may be a wireless or wired transmission module.

8. The reflective infrared ray artery wave as claimed in claim 7, wherein said wireless transmission module is a bluetooth module.

9. The reflective infrared ray artery wave as claimed in claim 7, wherein said wired transmission module is an USB transmission line.

10. The reflective infrared ray artery wave as claimed in claim 1 further comprising an infrared ray probe and a transmission module, wherein one end of said transmission module is electrically connected with said collecting module, and said infrared ray probe is electrically connected to another end of said transmission module by means of a transmission line.
